(19) United States
(12) Patent Application Publication
(10) Pub. No.: US 2025/0188003 A1
(43) Pub. Date: Jun. 12, 2025

(54) ETHYLENE OLIGOMERIZATION METHOD, AND ETHYLENE OLIGOMER THEREOF

(71) Applicant: HANWHA TOTALENERGIES PETROCHEMICAL CO., LTD., Chungcheongnam-do (KR)

(72) Inventors: Kye Sung CHO, Chungcheongnam-do (KR); Dong Geun LEE, Chungcheongnam-do (KR); Sang Joon OH, Chungcheongnam-do (KR); Ho Sik CHANG, Chungcheongnam-do (KR); Jin Suk LEE, Chungcheongnam-do (KR)

(21) Appl. No.: 18/686,239

(22) PCT Filed: Sep. 23, 2022
(86) PCT No.: PCT/KR2022/014241

§ 371 Date: Feb. 23, 2024

(30) Foreign Application Priority Data
Dec. 29, 2021 (KR) 10-2021-0190688

(51) Int. Cl. C07C 2/32; B01J 31/24; B01J 31/26; B01J 37/04; C07C 11/02

(52) U.S. Cl. CPC C07C 2/32; B01J 31/2409; B01J 31/26; B01J 37/04; C07C 11/02; B01J 2231/20; B01J 2531/004; B01J 2531/62; C07C 2531/14

(57) ABSTRACT

The present invention provides: an ethylene oligomerization method in which an ethylene oligomer is produced by reacting a chromium complex and an organic aluminium compound with ethylene; and the ethylene oligomer thereof.

### FIELD

The present invention relates to an ethylene oligomerization method and ethylene oligomers thereof.

### BACKGROUND

Among ethylene oligomers, 1-hexene and 1-octene are substances used in large quantities as comonomers in the polymerization of polyolefins, such as polyethylene and, as the production of polyolefins using homogeneous metallocene catalysts is increasing, the demand is steadily increasing.

Conventionally, as ethylene is oligomerized in the shell higher olefin process (SHOP) based on a nickel catalyst, various 1-alkenes with about 4 to about 30 carbon atoms are produced, from which 1-hexene and/or 1-octene may be separated and obtained.

Afterwards, a catalyst system was developed that could produce 1-hexene or 1-octene in high yield by increasing the selectivity in the ethylene oligomerization reaction.

As a representative example, U.S. Pat. No. 7,511,183B2 discloses a catalyst preparation method capable of selectively producing 1-octene and 1-hexene using a chromium trivalent compound (CrCl3 or Cr(acac)3), a bisphosphine ligand (iPrN(PPh2)2), and methylaluminoxane (MAO).

However, it was reported that the catalyst system required the use of a large amount of expensive MAO (Al/Cr=300-500) to achieve a commercially usable activity level, and suffered from production of a large amount of polyethylene (PE), which seriously impeded process stability (Organometallics, 27, 5712-5716).

Further, at high temperatures, the catalytic activity of the oligomerization catalyst system decreases, resulting in a decrease in the production and selectivity of olefins, especially 1-octene, and as the production of by-products increases, tube clogging and fouling occur, which inevitably leads to process interruption. This causes serious problems in the olefin polymerization process.

Specifically, polyethylene produced as a by-product forms a polymer layer, and a polymer layer is formed again on the formed polymer layer, lowering the flow rate of the fluid, and the polymer coating layer formed along the reactor wall serves as an insulator that negatively affects heat transfer. In other words, tube clogging and fouling occur, requiring a secondary process to remove the polymer layer, with the result of frequent process shutdowns.

Therefore, there is a need for an enhanced ethylene oligomerization method that may not only produce 1-hexene and 1-octene with high selectivity without reducing catalyst activity, but also significantly reduce the amount of polyethylene produced, which impedes process stability.

### DETAILED DESCRIPTION OF THE INVENTION

### Technical Problem

The present invention provides an ethylene oligomerization method that may not only produce 1-hexene and 1-octene with high selectivity, but also significantly reduce the amount of polyethylene produced, which impedes process stability, without reducing catalyst activity, and an ethylene oligomer.

### Technical Solution

The present invention provides an ethylene oligomerization method that suppresses polyethylene production while maintaining enhanced catalytic activity, and particularly increases selectivity for 1-octene, and an ethylene oligomer.

The present invention provides an ethylene oligomerization method comprising the step of producing an ethylene oligomer by reacting a chromium complex represented by chemical formula 1 below and an organoaluminium compound represented by chemical formula 2 below with ethylene at less than 60° C. in the presence of an organic solvent.

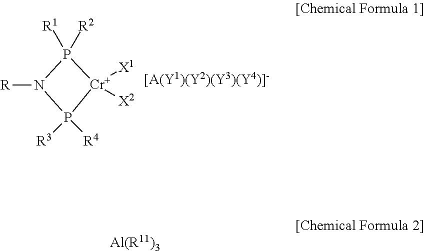

In chemical formula 1 and chemical formula 2,
R is a C1-C60 alkyl or a C6-C60 aryl, a C2-C60 heteroaryl containing at least one heteroatom selected from the group consisting of O, N, S, SI, and P; R¹ to R⁴ are independently a C1-C60 alkyl or a C6-C60 aryl, a C2-C60 heteroaryl containing at least one heteroatom selected from the group consisting of O, N, S, SI, and P; X¹ and X² each are independently a C1-C30 hydrocarbyl containing one or more selected from halogen, a C1-C30 alkyl, a C1-C30 alkylcarboxylate, acetylacetonate, ether, and amino; A is boron or aluminum; Y¹ to Y⁴ are independently a fluorine-substituted C6-C60 aryl, a fluorine-substituted C2-C60 heteroaryl containing at least one heteroatom selected from the group consisting of O, N, S, SI, and P, a fluorine-substituted C6-C60 aryloxy, or a fluorine-substituted C6-C60 alkoxy; and R¹¹ may be a C4-C8 alkyl.

In this case, in R, R¹ to R⁴, X¹, X², Y¹ to Y⁴, and R¹¹, the alkyl or aryl, heteroaryl may be further substituted with one or more selected from a C1-C30 alkyl, a C6-C30 aryl, a C1-C30 alkoxy, a monoC1-C30 alkylamino, a diC1-C30 alkylamino, a triC1-C30 alkylamino, a monoC6-C30 arylamino, a diC6-C30 arylamino, a triC6-C30 arylamino, a monoC1-C30 alkylsilyl, a diC1-C30 alkylsilyl, a triC1-C30 alkylsilyl, a monoC6-C30 arylsilyl, a diC6-C30 arylsilyl, and a triC6-C30 arylsilyl.

Further, the present invention provides an ethylene oligomer prepared by reacting a chromium complex represented by the above-described chemical formula 1 and an organoaluminium compound represented by the above-described chemical formula 2 with ethylene.

### Advantageous Effects

An ethylene oligomerization method according to an embodiment of the present invention may not only produce 1-hexene and 1-octene with high selectivity, but also significantly reduce the amount of polyethylene produced, which impedes process stability, without reducing catalyst activity.

### BRIEF DESCRIPTION OF THE DRAWINGS

FIG. 1 is a photograph of a polymer produced after a reaction according to embodiment 1 of the present invention; and

FIG. 2 is a photograph of a polymer produced after a reaction according to comparative example 1 of the present invention.

### MODE FOR CARRYING OUT THE INVENTION

Hereinafter, embodiments of the disclosure are described in detail with reference to the accompanying drawings. In assigning reference numerals to components of each drawing, the same components may be assigned the same numerals even when they are shown on different drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the present invention. These denotations are provided merely to distinguish a component from another, and the essence, order, or number of the components are not limited by the denotations.

In describing the positional relationship between components, when two or more components are described as “connected”, “coupled” or “linked”, the two or more components may be directly “connected”, “coupled” or “linked”, or another component may intervene. Here, the other component may be included in one or more of the two or more components that are “connected”, “coupled” or “linked” to each other.

When such terms as, e.g., “after”, “next to”, “after”, and “before”, are used to describe the temporal flow relationship related to components, operation methods, and fabricating methods, it may include a non-continuous relationship unless the term “immediately” or “directly” is used.

When a component is designated with a value or its corresponding information (e.g., level), the value or the corresponding information may be interpreted as including a tolerance that may arise due to various factors (e.g., process factors, internal or external impacts, or noise).

As used herein, the term “alkyl” refers to a saturated straight-chain or branched non-cyclic hydrocarbon having (unless the number of carbon atoms is specifically limited) 1 to 60 carbon atoms, preferably 1 to 30 carbon atoms, preferably 1 to 20 carbon atoms in an aspect, preferably 1 to 10 carbon atoms in an aspect, or preferably 1 to 7 carbon atoms in an aspect. “Lower alkyl” refers a straight-chain or branched alkyl having 1 to 7 carbon atoms, preferably 1 to 5 carbon atoms in an aspect. Representative saturated straight-chain alkyls include-methyl, -ethyl, -n-propyl, -n-butyl, -n-pentyl, -n-hexyl, -n-heptyl, -n-octyl, -n-nonyl, and -n-decyl, while the saturated branched alkyls include-isopropyl, -sec-butyl, -isobutyl, -tert-butyl, isopentyl, 2-methylhexyl, 3-methylbutyl, 2-methylpentyl, 3-methylpentyl, 4-methylpentyl, 2-methylhexyl, 3-methylhexyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-dethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl.

“C1-C10” in the disclosure means that the number of carbon atoms is 1 to 10. For example, C1-C10 alkyl means an alkyl having 1 to 10 carbon atoms.

As used herein, the terms “halogen” and “halo” mean fluorine, chlorine, bromine or iodine.

As used herein, the term “fluorine-substituted aryl”, “fluorine-substituted aryloxy” or “fluorine-substituted alkoxy” refers to an aryl, aryloxy or alkoxy group in which one or more hydrogen atoms are substituted with a fluorine atom. For example, haloaryl includes —C₆H₄F, —C₆H₃F₂, —C₆HF₄, or the like. Here, aryl and halogen are as defined above, and alkoxy is as defined below.

As used herein, the term “alkoxy” refers to —O-(alkyl) including-OCH₃, —OCH₂CH₃, —O(CH₂)₂CH₃, —O(CH₂)₃CH₃, —O(CH₂)₄CH₃, —O(CH₂)₅CH₃ or the like, where the alkyl is as defined above.

As used herein, the term “lower alkoxy” refers to —O-(lower alkyl), where lower alkyl is as defined above.

As used herein, the term “aryl” refers to a carbocyclic aromatic group containing 5 to 10 ring atoms. Representative examples include phenyl, tolyl, xylyl, naphthyl, tetrahydronaphthyl, anthracenyl, fluorenyl, indenyl, azulenyl, or the like but are not limited thereto. The carbocyclic aromatic group may be optionally substituted.

As used herein, the term “aryloxy” means RO—, where R is aryl as defined above. “Arylthio” is RS—, where R is aryl as defined above.

As used herein, the term “mono-alkylamino” refers to —NH(alkyl) including —NHCH₃, —NHCH₂CH₃, —NH(CH₂)₂CH₃, —NH(CH₂)₃CH₃, —NH(CH₂)₄CH₃, —NH(CH₂)₅CH₃ or the like, where alkyl is as defined above.

As used herein, the term “di-alkylamino” refers to —N(alkyl)(alkyl) including —N(CH₃)₂, —N(CH₂CH₃)₂, —N((CH₂)₂CH₃)₂, —N(CH₃)(CH₂CH₃), or the like, where each alkyl is independently an alkyl as defined above.

As used herein, the term “mono-alkylsilyl” refers to —SiH₂(alkyl) including SiH₂CH₃, —SiH₂CH₃, —SiH₂(CH₂)₂CH₃, —SiH₂(CH₂)₃CH₃, —SiH₂(CH₂)₄CH₃, —SiH₂(CH₂)₅CH₃, or the like, where alkyl is as defined above.

As used herein, the term “di-alkylsilyl” refers to SiH(alkyl)(alkyl) including —SiH(CH₃)₂, —SiH(CH(CH₃)₂)(CH₃), —SiH((CH₂)₂CH₃)₂, —SiH(CH₃)(CH₂CH₃), or the like, where each alkyl is independently an alkyl as defined above.

As used herein, the term “trialkylsilyl” refers to Si(alkyl)(alkyl)(alkyl) including —Si(CH₃)₃, —Si(CH₂(CH₃))₃, —Si((CH₂)₂CH₃)₃, —Si(CH₃)₂(CH₂CH₃), or the like, where each alkyl is independently an alkyl as defined above.

As used herein, the terms “monoarylsilyl”, “diarylsilyl”, and “triarylsilyl” refer to a substituent corresponding to aryl instead of alkyl in “monoalkylsilyl”, “dialkylsilyl”, or “trialkylsilyl”.

As used herein, the term “heteroatom” refers to N, O, S, P or Si unless otherwise specified, but is not limited thereto.

Hereinafter, an ethylene oligomerization method according to an embodiment of the present invention is described in detail.

An ethylene oligomerization method according to the present invention provides an ethylene oligomerization method comprising the step of producing an ethylene oligomer by reacting a chromium complex represented by chemical formula 1 below and an organoaluminium compound represented by chemical formula 2 below with ethylene at less than 60° C. in the presence of an organic solvent.

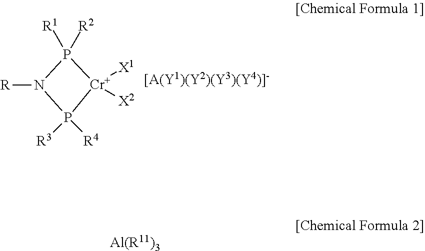

In chemical formula 1 and chemical formula 2,
R is a C1-C60 alkyl or a C6-C60 aryl, a C2-C60 heteroaryl containing at least one heteroatom selected from the group consisting of O, N, S, SI, and P;
R¹ to R⁴ are independently a C1-C60 alkyl or a C6-C60 aryl, a C2-C60 heteroaryl containing at least one heteroatom selected from the group consisting of O, N, S, SI, and P;
X¹ and X² are each independently a C1-C30 hydrocarbyl containing one or more selected from halogen, a C1-C30 alkyl, a C1-C30 alkylcarboxylate, acetylacetonate, or ether, and amino;
A is boron or aluminum;
Y¹ to Y⁴ are independently a fluorine-substituted C6-C60 aryl, a fluorine-substituted C₂-C₆₀ heteroaryl containing at least one heteroatom selected from the group consisting of O, N, S, SI, and P, a fluorine-substituted C6-C60 aryloxy, or a fluorine-substituted C6-C60 alkoxy;
R¹¹ may be a C4-C8 alkyl.

In this case, in R, R¹ to R⁴, X¹, X², Y¹ to Y⁴, and R¹¹, the alkyl or aryl, heteroaryl may be further substituted with one or more selected from a C1-C30 alkyl, a C6-C30 aryl, a C1-C30 alkoxy, a monoC1-C30 alkylamino, a diC1-C30 alkylamino, a triC1-C30 alkylamino, a monoC6-C30 arylamino, a diC6-C30 arylamino, a triC6-C30 arylamino, a monoC1-C30 alkylsilyl, a diC1-C30 alkylsilyl, a triC1-C30 alkylsilyl, a monoC6-C30 arylsilyl, a diC6-C30 arylsilyl, and a triC6-C30 arylsilyl.

The ethylene oligomerization method according to the present invention may have excellent catalytic activity by using the chromium complex represented by chemical formula 1 and the organoaluminium compound represented by chemical formula 2, thereby preparing ethylene oligomers with excellent selectivity and conversion rate even at low temperatures.

Further, the ethylene oligomerization method according to the present invention uses a specific chromium complex represented by chemical formula 1, thus exhibiting high catalytic activity without using conventional expensive methylaluminoxane and enabling ethylene oligomerization.

Preferably, the step of preparing the ethylene oligomer according to an embodiment of the present invention may further include hydrogen. By further including hydrogen in the step of preparing the ethylene oligomer according to an embodiment of the present invention, it is possible to maintain catalytic activity and process stability by dramatically suppressing the production of by-products, such as polyolefin.

Preferably, an ethylene oligomerization method according to an embodiment of the present invention may comprise the steps of reacting a chromium complex represented by chemical formula 1 with an organoaluminium compound represented by chemical formula 2 in the presence of an organic solvent; and sequentially injecting hydrogen and ethylene into the mixture of the step. By sequentially adding hydrogen and ethylene after the step of reacting the chromium complex represented by chemical formula 1 and the organoaluminium compound represented by chemical formula 2, it is possible to increase the catalyst activity while dramatically decreasing the production of reaction by-products, such as polyethylene.

In terms of enhancing selectivity and catalytic activity, the molar ratio of ethylene and hydrogen according to an embodiment of the present invention may be 1:0.001 to 3.0, preferably 1:0.01 to 2.0, more preferably 1:0.04 to 0.32, more preferably 0.09 to 0.19.

The ethylene oligomerization reaction according to an embodiment of the present invention may be performed at above 30° C. to below 60° C., preferably above 30° C. to below 50° C., and the reaction time may be 10 minutes to 2 hours, preferably 10 minutes to 1 hour.

The ethylene oligomerization method according to an embodiment of the present invention may be performed in any reactor, but may be performed preferably in a continuous stirred tank reactor (CSTR) or a tubular reactor (PFR), and more preferably a continuous stirred tank reactor (CSTR).

When the reactor according to an embodiment of the present invention is a continuous stirred tank reactor, it is preferable that 80% or less, preferably 50% or less, of the total volume of the reactor is maintained in the liquid phase.

The molar ratio of the chromium complex and the organoaluminum compound according to an embodiment of the present invention may be 1:10 to 500, preferably 1:100 to 300, and more preferably 1:150 to 250.

As a more preferable combination of chemical formula 1 and chemical formula 2 according to an embodiment of the present invention, in chemical formula 1 and chemical formula 2 of the present invention, R may be a C1-C60 alkyl; R¹ to R⁴ may be independently a C6-C60 aryl; X¹ and X² may be each independently a C1-C30 hydrocarbyl containing halogen, a C1-C30 alkyl, a C1-C30 alkylcarboxylate, acetylacetonate, or ether; A may be boron or aluminum; Y¹ to Y⁴ may be independently a fluorine-substituted C6-C60 aryl, a fluorine-substituted C6-C60 aryloxy, or a fluorine-substituted C6-C60 alkoxy; and R¹¹ to R¹³ may be independently a C1-C20 alkyl.

In this case, the alkyl of R and the aryl of R¹ to R⁴ may be one that may be further substituted with one or more selected from a C1-C30 alkyl, a C6-C30 aryl, a C1-C30 alkoxy, a monoC1-C30 alkylamino, a diC1-C30 alkylamino, a triC1-C30 alkylamino, a monoC6-C30 arylamino, a diC6-C30 arylamino, a triC6-C30 arylamino, a monoalkylsilyl, a diC1-C30 alkylsilyl, a triC1-C30 alkylsilyl, a monoC6-C30 arylsilyl, a diC6-C30 arylsilyl and a triC6-C30 arylsilyl.

As a more preferable combination of chemical formula 1 and chemical formula 2 according to an embodiment of the present invention, in chemical formula 1 and chemical formula 2, R may be a C1-C30 alkyl; R¹ to R⁴ may be independently a C6-C30 aryl; X¹ and X² may be each independently a C1-C20 hydrocarbyl containing halogen, a C1-C30 alkyl, a C1-C30 alkylcarboxylate, acetylacetonate, or ether; A may be boron; Y¹ to Y⁴ may be independently a fluorine-substituted C6-C30 aryl, a fluorine-substituted C6-C30 aryloxy, or a fluorine-substituted C6-C30 alkoxy; and R¹¹ to R¹³ may be independently a C1-C20 alkyl.

In this case, the alkyl of R and the aryle of R¹ to R⁴ may be one that may be further substituted with one or more selected from a C1-C20 alkyl, a C6-C20 aryl, a C1-C20 alkoxy, a monoC1-C20 alkylamino, a diC1-C20 alkylamino, a triC1-C20 alkylamino, a monoC6-C20 arylamino, a diC6-C20 arylamino, a triC6-C20arylamino, a monoC1-C20alkylsilyl, a diC1-C20alkylsilyl, a triC1-C20alkylsilyl, a monoC6-C20arylsilyl, a diC6-C20arylsilyl, and a triC6-C20aryl.

More preferably, R may be a C1-C20 alkyl; R¹ to R⁴ may be independently a C6-C20 aryl; X¹ and X² may be each independently halogen, a C1-C20 alkyl, a C1-C20 alkylcarboxylate or acetylacetonate; A may be boron; Y¹ to Y⁴ may be independently a fluorine-substituted C6-C20 aryl, a fluorine-substituted C6-C20 aryloxy, or a fluorine-substituted C6-C20 alkoxy; R¹¹ to R¹³ may be independently a C1-C10 alkyl, and the alkyl of R and the aryle of R¹ to R⁴ may be one that may be further substituted with one or more selected from a C1-C10 alkyl, a C6-C12 aryl, a C1-C10 alkoxy, a monoC1-C10 alkylamino, a diC1-C10 alkylamino, a triC1-C10 alkylamino, a monoC6-C12 arylamino, a diC6-C12 arylamino, a triC6-C12 arylamino, a monoC1-C10 alkylsilyl, a diC1-C10 alkylsilyl, and a triC1-C10 alkylsilyl.

More preferably, R may be a C1-C10 alkyl; R¹ to R⁴ may be independently a C6-C12aryl; X¹ and X² may be each independently halogen; A may be boron; Y¹ to Y⁴ may be independently a fluorine-substituted C6-C12 aryl; and R¹¹ to R¹³ may be independently a C1-C7 alkyl.

In this case, the alkyl of R and the aryl of R¹ to R⁴ may be one that may be further substituted with one or more selected from a C1-C7 alkyl and a triC1-C7 alkylsilyl.

In terms of catalytic efficiency, selectivity of ethylene oligomers, and inhibition of polyolefin production, chemical formula 1 may be more preferably represented as chemical formula 3 below.

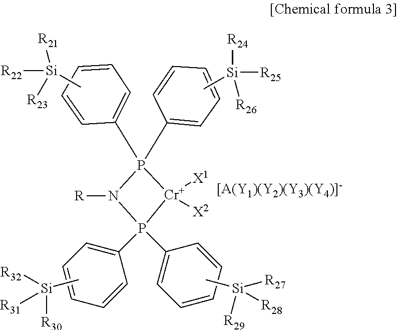

in chemical formula 3,
R may be a C1-C30 alkyl or a C6-C30 aryl;
X¹ and X² may be each independently a C1-C20 hydrocarbyl containing halogen, a C1-C20 alkyl, a C1-C20 alkylcarboxylate, acetylacetonate or ether;
A may be boron or aluminum;
Y¹ to Y⁴ may be independently a fluorine-substituted C6-C30 aryl, a fluorine-substituted C6-C30 aryloxy, or a fluorine-substituted C6-C30 alkoxy;
R²¹ to R³² may be independently a C1-C20 alkyl,

The alkyl or aryl of R may be further substituted with one or more selected from a C1-C20 alkyl, a C6-C20 aryl, a C1-C20 alkoxy, a monoC1-C20 alkylamino, a diC1-C20 alkylamino, a triC1-C20 alkylamino, a monoC1-C20 arylamino, a diC6-C20 arylamino, and a triC6-C20 arylamino.

The chromium complex represented by chemical formula 3 according to an embodiment of the present invention has further enhanced catalytic activity and ethylene oligomer selectivity by introducing a trialkylsilyl group, a specific substituent, to phenyl (Ph) bonded to phosphorus (P).

Preferably, in chemical formula 3 according to an embodiment of the present invention, R may be a C1-C20 alkyl; X¹ and X² may be each independently halogen, a C1-C20 alkyl, a C1-C20 alkylcarboxylate, and acetylacetonate; A may be boron; Y¹ to Y⁴ may be independently a fluorine-substituted C6-C20 aryl or a flurine-substituted C6-C20 aryloxy, preferably R may be a C1-C10 alkyl; X¹ and X² may be each independently halogen or a C1-C10 alkyl; A may be boron; Y¹ to Y⁴ may be independently a fluorine-substituted C6-C12 aryl.

More preferably, in chemical formula 3 according to an embodiment of the present invention, R may be a C₁-C₅ alkyl; X¹ and X² may be chlorine; A may be boron; Y¹ to Y⁴ may be (C₆F₅)₄; and R²¹ to R³² may be independently a C5-C8 alkyl.

Preferably, chemical formula 3 according to an embodiment of the present invention may be represented as chemical formula 4 below.

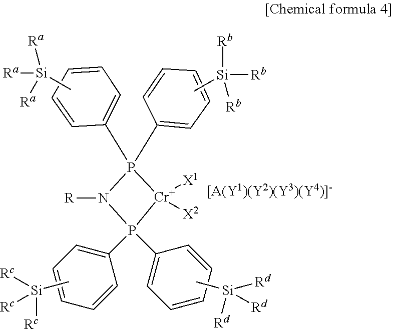

in chemical formula 4,
R may be a C1-C20 alkyl or a C6-C20 aryl;
X¹ and X² may be each independently a C1-C20 hydrocarbyl containing halogen, a C1-C20 alkyl, a C1-C20 alkylcarboxylate, acetylacetonate or ether;
A may be boron or aluminum;
Y¹ to Y⁴ may be independently a fluorine-substituted C6-C20 aryl, a fluorine-substituted C6-C20 aryloxy, or a fluorine-substituted C6-C20 alkoxy;
R^{a} to R^{d} may be independently a C1-C10 alkyl,

The alkyl or aryl of R may be further substituted with one or more selected from C1-C20 alkyls.

Preferably, in chemical formula 4 according to an embodiment of the present invention, R may be a C1-C10 alkyl; X¹ and X² may be each independently halogen, a C1-C10 alkyl or acetylacetonate; A may be boron; Y¹ to Y⁴ may be independently a fluorine-substituted C6-C12 aryl; R^{a} to R^{d} may be independently a C1-C8 alkyl, and the alkyl or aryl of R may be further substituted with one or more selected from C1-C8 alkyls, more preferably R^{a} to R^{d} may all be a C1-C8 alkyl.

Preferably, in an embodiment of the present invention, in the chromium complex of chemical formula 4, R may be a C₁-C₅ alkyl, R^{a} to R^{d} may be a C5-C8 alkyl, more preferably R may be a branched chain C₃-C₅ alkyl, and R^{a} to R^{d} may be a C5-C8 alkyl, more preferably R may be an isopropyl group, and R^{a} to R^{d} may be an octyl group (n-Octyl), [A(Y¹)(Y²)(Y³)(Y⁴)]⁻ may be [B(C₆F₅)₄]. The chromium complex in such a structural example may significantly enhance the activity and selectivity when applied to a catalyst system for ethylene oligomerization reaction while further reducing the amount of high molecular weight polyethylene compound produced as a by-product.

In the ethylene oligomerization method according to an embodiment of the present invention, the type of organic solvent is not particularly limited, but may be a hydrocarbon solvent substituted or unsubstituted with halogen. Specifically, the hydrocarbon solvent may be an aliphatic hydrocarbon solvent having 4 to 20 carbon atoms, an aromatic hydrocarbon solvent having 6 to 20 carbon atoms, or a mixture thereof. More specifically, examples of the hydrocarbon solvent substituted or unsubstituted with halogen may include toluene, xylene, chlorobenzene, dichlorobenzene, dichloromethane, methylcyclohexene, cyclohexene, etc., preferably dichloromethane, methylcyclohexene, and cyclohexene. When the solvents exemplified above are used, polymerization activity is high, and it is easy to separate the solvent from 1-hexene and 1-octene, which are products after the oligomerization reaction.

An ethylene oligomer according to another embodiment of the present invention provides an ethylene oligomer prepared by the ethylene oligomer according to an embodiment of the present invention described above.

For example, the ethylene oligomer according to another embodiment of the present invention may be an ethylene olygomer prepared by reacting a chromium complex represented by the above-described chemical formula 1 and an organoaluminium compound represented by the above-described chemical formula 2 with ethylene.

In this case, the ethylene oligomer according to another embodiment of the present invention may have a polyethylene content of 0.3 wt % or less, preferably 0.2 wt % or less, and more preferably less than 0.1%.

The ethylene oligomerization method according to an embodiment of the present invention may have high activity and selectivity and may maintain process stability by suppressing the production of polyethylene by using a specific chromium complex, cocatalyst, and hydrogen additive.

Hereinafter, the present invention is described in greater detail with reference to embodiments thereof. However, the embodiments are for illustrative purposes only and the present invention is not limited thereto.

### Preparation Example 1

A chromium complex represented by chemical formula A below was prepared according to the following method.

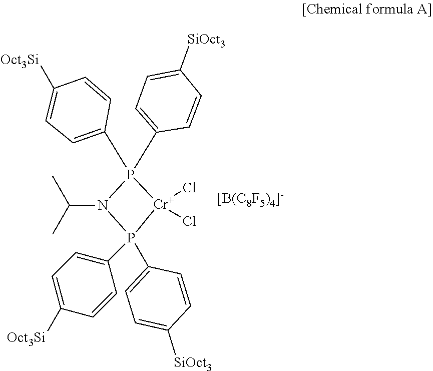

### Synthesis of Cl—Si(n-Octyl)₃

A solution of acetyl chloride (1.54 g, 19.6 mmol) dissolved in CH₂Cl₂ (7 mL) was added dropwise to a CH₂Cl₂ (20 mL) solution containing trioctylsilane (4.83 g, 13.1 mmol) and FeCl₃ (0.0549 g, 0.262 mmol). After identifying that the solution turned into yellow as FeCl₃ was dissolved, the solution was stirred at room temperature for 24 hours. The solvent, by-product acetaldehyde, and non-reactant acetyl chloride were removed using a vacuum line. The residue was dissolved in hexane (10 mL), and an insoluble brown solid (FeCl₃) was removed by filtration (Celite-aided filtration). The solvent was removed using a vacuum line, and a light yellow oil compound was obtained (4.98 g, 98%).

### Synthesis of BrC₆H₄-p-Si(n-Octyl)₃

1,4-dibromobenzene (3.31 g, 14.0 mmol) was dissolved in THF (35 mL), and n-butyllithium (5 mL, 2.5 M hexane solution, 12.5 mmol) was injected at −78° C., and then was maintained at −78° C. while being stirred for 2 hours. Cl—Si(n-Octyl)₃ (4.79 g, 11.9 mmol) was dissolved in THF (6 mL) and injected and, after increasing the temperature to room temperature, was left to react for 3 hours. The solvent was removed using a vacuum line, the desired product was dissolved in hexane (18 mL), and the insoluble white solid (LiBr) was then removed through filtration (Celite-aided filtration). The solvent was removed from the filtered liquid using a vacuum line, and was dissolved in hexane (18 mL) and passed through a short pad of silica gel (6.22 g). After the solvent was removed using a vacuum line, the residue was vacuum distilled at 80° C. to remove non-reactants (1,4-dibromobenzene), and the target compound was obtained as an oil (5.71 g, 92%).

### Synthesis of CIP[C₆H₄-p-Si(n-Octyl)₃]₂

BrC₆H₄-p-Si(n-Octyl)₃ (5.71 g, 10.9 mmol) was dissolved in THF (39 mL), and n-butyllithium (4.36 mL, 2.5 M hexane solution, 10.9 mmol) was injected at −78° C., and then was maintained at −78° C. while being stirred for 1 hour. Dichloro(diethylamino)phosphine (0.949 g, 5.45 mmol) dissolved in THF (9 mL) was injected for 15 minutes, and the temperature was raised to 5° C. and maintained while leaving it to react for 2 hours. After injecting methylcyclohexane (19 mL), the solvent was removed using a vacuum line at room temperature, and methylcyclohexane (31 mL) was added and insoluble white solids (LiBr and LiCl) were removed by filteration (Celite-aided filtration). After removing the solvent, PCI₃ (4.12 g, 30.0 mmol) was added and left to react at 70° C. for 2 hours. By vacuum distillation at 80° C., PCl, a non-reactive substance, and the by-product, dichloro(diethylamino)phosphine, were removed, obtaining the yellow oil compound. The oil compound was dissolved in hexane (23 mL), and insoluble by-products were removed by filtration (Celite-aided filtration). The solvent was removed through a vacuum line, and a yellow oil compound was obtained as the target compound (5.18 g, 99%).

### Synthesis of i-PropylN[P(C₆H₄-p-Si(n-Octyl)₃)₂]₂

A solution of i-PrNH₂ (0.135 g, 2.28 mmol) dissolved in CH₂Cl₂ (11 mL) was added dropwise to a CH₂Cl₂ (19 mL) solution containing CIP[C₆H₄-p-Si(n-Octyl)₃]₂ (4.79 g, 5.02 mmol) and Et₃N (2.31 g, 22.8 mmol). After the product was stirred at room temperature for 12 hours, volatile components were removed using a vacuum line. After adding hexane (40 mL) to the residue, the insoluble by-product (Et₃NH)⁺Cl⁻ was removed by filtration (Celite-aided filtration). The filtered liquid was passed through a short pad of silica gel pre-processed with hexane/Et₃N (v/v, 50:1), and the solvent was then removed using a vacuum line, obtaining a colorless oil product (4.26 g, 98%).

### Synthesis of [(i-PropylN[P(C₆H₄-p-Si(n-Octyl)₃)₂]₂)-CrCl₂]⁺[B(C₆F₅)₄]

A solution of i-PropylN[P(C₆H₄-p-Si(n-Octyl)₃)₂]₂ (1.41 g, 0.742 mmol) dissolved in CH₂Cl₂ (13 mL) was added dropwise to a solution obtained by dissolving [CrCl₂(NCCH₃)₄]⁺[B(C₆F₅)₄] (0.715 g, 0.742 mmol) in CH₂Cl₂ (4.5 mL). After the product was stirred at room temperature for 2.5 hours, the solvent was removed using a vacuum line, obtaining a viscous green oil.

The obtained oil was dissolved in methylcyclohexane (5 mL) and the solvent was removed using a vacuum line. This process was repeated until CH₃CN and CH₃Cl₂ were completely removed, obtaining a viscous green oil (2 g, 100%). The so-obtained product was dissolved in methylcyclohexane (23.4 mL) to make a 10 wt % solution and used for ethylene oligomerization.

### [Embodiment 1] Ethylene Oligomerization Reaction

After adding 200 ml of methylcyclohexane and 100 mol of trinoctylaluminum (TnOA) to a 500 ml autoclave reactor heated to 40° C., the chromium complex (1.5 mg, 0.6 mol) of chemical formula A prepared in Preparation Example 1 was injected into the reactor using a syringe. After hydrogen was injected at a pressure of 80 psig, ethylene gas was injected at a pressure of 500 psig. After being left to react for 30 minutes while maintaining the reaction temperature at 40° C. while continuously supplying ethylene to maintain the internal pressure of the reactor at 500 psig, the reactor was cooled, and ethylene gas was discharged, terminating the reaction.

For product analysis, the content of the produced olygomer {1-octene (1-C8), 1-hexene (1-C6), methylcyclopentane+methylenecyclopentane (cy-C6), and higher oligomers above C10 (>C10)} was measured through gas chromatography (GC analysis), calculating the weight ratio of the product. The produced solid-state polyethylene was separated through filtration at room temperature and its weight was measured, and the wt % of polyethylene was calculated through the equation [weight (g) of produced PE/total weight (g) of product].

### [Comparative Example 1] Ethylene Oligomerization Reaction

The ethylene oligomerization reaction was performed in the same manner as in embodiment 1, except that triisobutylaluminum (TiBA) was used instead of trinoctylaluminum (TnOA) as the cocatalyst.

The activity level of the olefin polymerization reaction of embodiment 1 and comparative example 1 and the composition of the prepared polymer were shown in Table 1 below.

TABLE 1

| | | Activity | 1-C8 | 1-C6 | Cy-C6 | C10+ | PE |
|---|---|---|---|---|---|---|---|
| | cocatalyst | (kg/g-Cr/hr) | (wt %) | (wt %) | (wt %) | (wt %) | (wt %) |
| Embodiment 1 | TnOA | 11,872 | 67.2 | 13.3 | 5.3 | 14.2 | 0.04 |
| comparative | TiBA | 10,689 | 68.0 | 13.2 | 5.4 | 13.3 | 0.2 |
| example 1 | | | | | | | |

As shown in Table 1, in the case of embodiment 1 in which TnOA is used as the cocatalyst, it may be identified that the activity is superior, and the amount of polyethylene by-product production is reduced by more than 80% as compared with comparative example 1 that uses TiBA.

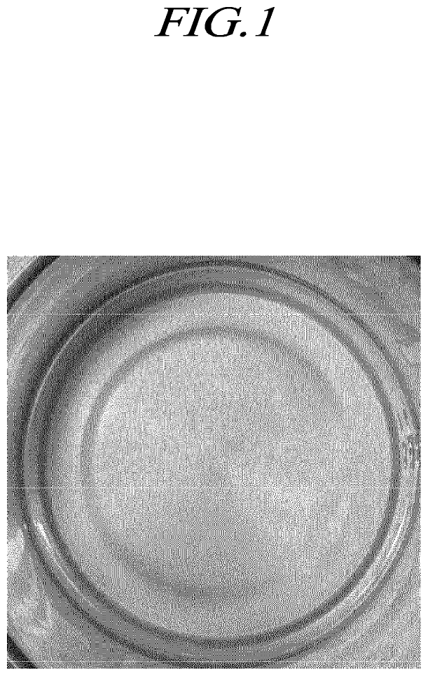

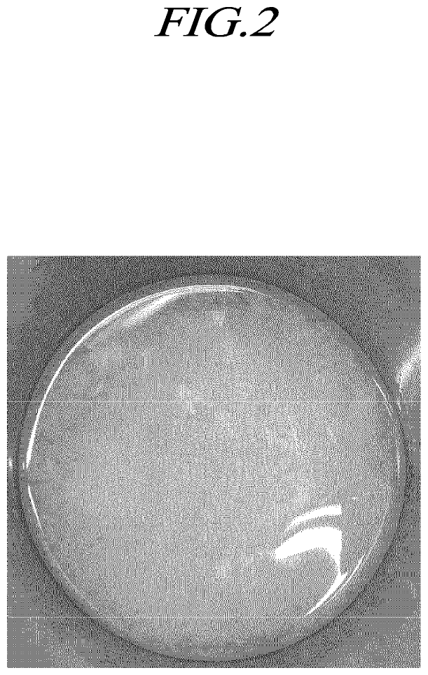

A look at FIGS. 1 and 2 reveals a noticeable difference in polyethylene accumulated in the reactor after completion of the reaction.

The ethylene oligomerization method according to an embodiment of the present invention may produce ethylene oligomers with high catalytic activity, excellent selectivity and conversion rate by performing the oligomerization reaction with a specific catalyst and cocatalyst at a controlled temperature and enable mass production by performing ethylene oligomerization even at low temperatures, which is very advantageous for commercialization.

Further, the ethylene oligomerization method according to an embodiment of the present invention may significantly reduce the amount of polyethylene produced and maintain process stability by preventing tube clogging and fouling and is thus very economical.

Further, the ethylene oligomerization method according to an embodiment of the present invention does not use conventional expensive methylaluminoxane but uses a chromium complex represented by chemical formula 1 described above, and is thus very economical, and may provide excellent catalytic activity, enabling ethylene oligomerization at low temperatures, and making it applicable to various reactors.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the disclosure should be construed by the following claims, and all technical spirits within equivalents thereof should be interpreted to belong to the scope of the disclosure.

### INDUSTRIAL AVAILABILITY

The present invention provides an ethylene oligomerization method that may not only produce 1-hexene and 1-octene with high selectivity, but also significantly reduce the amount of polyethylene produced, which impedes process stability, without reducing catalyst activity, and an ethylene oligomer.

1. An ethylene oligomerization method comprising the step of producing an ethylene oligomer by reacting a chromium complex represented by chemical formula 1 and an organoaluminium compound represented by chemical formula 2 with ethylene at less than 60° C. in the presence of an organic solvent, wherein 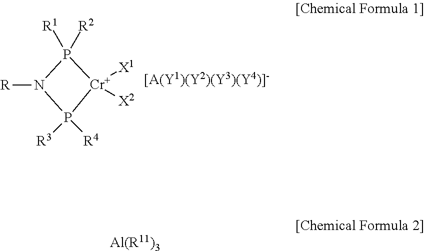
In chemical formula 1 and chemical formula 2,
R is a C1-C60 alkyl or a C6-C60 aryl, a C2-C60 heteroaryl containing at least one heteroatom selected from the group consisting of O, N, S, SI, and P;
R¹ to R⁴ are independently a C1-C60 alkyl or a C6-C60 aryl, a C2-C60 heteroaryl containing at least one heteroatom selected from the group consisting of O, N, S, SI, and P;
X¹ and X² are each independently a C1-C30 hydrocarbyl containing one or more selected from halogen, a C1-C30 alkyl, a C1-C30 alkylcarboxylate, acetylacetonate, or ether, and amino;
A is boron or aluminum;
Y¹ to Y⁴ are independently a fluorine-substituted C6-C60 aryl, a fluorine-substituted C2-C60 heteroaryl containing at least one heteroatom selected from the group consisting of O, N, S, SI, and P, a fluorine-substituted C6-C60 aryloxy, or a fluorine-substituted C6-C60 alkoxy;
R¹¹ is a C4-C8 alkyl,
wherein in R, R¹ to R⁴, X¹, X², Y¹ to Y⁴, and R¹¹, the alkyl or aryl, heteroaryl may be further substituted with one or more selected from a C1-C30 alkyl, a C6-C30 aryl, a C1-C30 alkoxy, a monoC1-C30 alkylamino, a diC1-C30 alkylamino, a triC1-C30 alkylamino, a monoC6-C30 arylamino, a diC6-C30 arylamino, a triC6-C30 arylamino, a monoC1-C30 alkylsilyl, a diC1-C30 alkylsilyl, a triC1-C30 alkylsilyl, a monoC6-C30 arylsilyl, a diC6-C30 arylsilyl, and a triC6-C30 arylsilyl.

2. The ethylene oligomerization method of claim 1, wherein R¹¹ in chemical formula 2 is an n-octyl group.

3. The ethylene oligomerization method of claim 1, wherein the reaction is performed at a temperature above 30° C. and below 60° C. for 10 minutes to 2 hours.

4. The ethylene oligomerization method of claim 1, wherein a molar ratio of the chromium complex and the organoaluminum compound is 1:10 to 300.

5. The ethylene oligomerization method of claim 1, wherein the step of preparing the ethylene oligomer includes hydrogen.

6. The ethylene oligomerization method of claim 5, wherein a molar ratio of ethylene and hydrogen is 1:0.001 to 3.0.

7. An ethylene oligomer prepared by reacting a chromium complex represented by chemical formula 1 and an organoaluminium compound represented by chemical formula 2 with ethylene, wherein 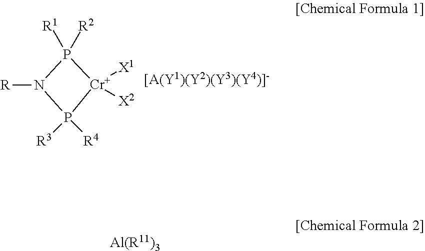
In chemical formula 1 and chemical formula 2,
R is a C1-C60 alkyl or a C6-C60 aryl, a C2-C60 heteroaryl containing at least one heteroatom selected from the group consisting of O, N, S, SI, and P;
R¹ to R⁴ are independently a C1-C60 alkyl or a C6-C60 aryl, a C2-C60 heteroaryl containing at least one heteroatom selected from the group consisting of O, N, S, SI, and P;
X¹ and X² are each independently a C1-C30 hydrocarbyl containing one or more selected from halogen, a C1-C30 alkyl, a C1-C30 alkylcarboxylate, acetylacetonate, or ether, and amino;
A is boron or aluminum;
Y¹ to Y⁴ are independently a fluorine-substituted C6-C60 aryl, a fluorine-substituted C₂-C60 heteroaryl containing at least one heteroatom selected from the group consisting of O, N, S, SI, and P, a fluorine-substituted C6-C60 aryloxy, or a fluorine-substituted C6-C60 alkoxy;
R¹¹ is a C4-C8 alkyl,
wherein in R, R¹ to R⁴, X¹, X², Y¹ to Y⁴, and R¹¹, the alkyl or aryl, heteroaryl may be further substituted with one or more selected from a C1-C30 alkyl, a C6-C30 aryl, a C1-C30 alkoxy, a monoC1-C30 alkylamino, a diC1-C30 alkylamino, a triC1-C30 alkylamino, a monoC6-C30 arylamino, a diC6-C30 arylamino, a triC6-C30 arylamino, a monoC1-C30 alkylsilyl, a diC1-C30 alkylsilyl, a triC1-C30 alkylsilyl, a monoC6-C30 arylsilyl, a diC6-C30 arylsilyl, and a triC6-C30 arylsilyl.

8. The ethylene oligomer of claim 7, wherein R¹¹ in chemical formula 2 is an n-octyl group.

9. The ethylene oligomer of claim 7, wherein the reaction is performed at a temperature above 30° C. and below 60° C. for 10 minutes to 2 hours.

10. The ethylene oligomer of claim 7, wherein a molar ratio of the chromium complex and the organoaluminum compound is 1:10 to 300.

11. The ethylene oligomer of claim 7, wherein the step of preparing the ethylene oligomer includes hydrogen.

12. The ethylene oligomer of claim 11, wherein a molar ratio of ethylene and hydrogen is 1:0.001 to 3.0.
